SEQUENCE LISTING (6 sequences)

SEQ ID NO: 1 - DNA, 883 bp, synthetic construct
  source 1..883: mol_type = other DNA
atggtctactatgccccttggacagaattcaaaaagaggaggagtaacgctattcatcccttaatgaagaagcagaaccatcaaaagaacactagacaagaagctgaattgctaccactagccatgcaagagcaaggaagcaaagaggtactctctctctctctctctctctctctctctgtttctctattgatcagttgtgagcacatgatatttgagatcaaacactgatgtcattactttaaggtcacgtcacgtatttattttcaaatttttatgtccatgatatatcaagattgtgacatatatttcatctaagctatcaagattgttggagagattgattcctagaatgagcttcaaggaattatgcaaggcaaccgattactttagcacagataacgttttagggataggaacgacagggattatgtacaaggcaaaggtaccaaataactgttttctagcagttaagagactatatggcgctgatgaatacaagagggaattcttgcttgaaacaatgattctggggagacacaggcacagaaacatagctccattggtaggattctgcatagaaaaaagagaaaggattttggtgtataagtacatgtctaatggaagactcagtgattggttccattctgatgaaggagatcaaaaaataaaactagaatggccagagagaattcacattgcacttgggattgcaagaggtttatcttggctccacaaaaggtgcaagatattccatcttaatttagattcagagtgtgtcttgctagacaggtattttgagccaaaaatatccaattttgagaaggcaaaatttctaaaccatacagttgaagatcatgtgaggatgaatgttatgaattag

SEQ ID NO: 2 - DNA, 20 bp, synthetic construct
  source 1..20: mol_type = other DNA
gagcttcaaggaattatgca

SEQ ID NO: 3 - DNA, 20 bp, synthetic construct
  source 1..20: mol_type = other DNA
gcacagaaacatagctccat

SEQ ID NO: 4 - DNA, 52 bp, synthetic construct
  source 1..52: mol_type = other DNA
tggtctcgtgcagagcttcaaggaattatgcagttttagagctagaaatagc

SEQ ID NO: 5 - DNA, 52 bp, synthetic construct
  source 1..52: mol_type = other DNA
tggtctcgaaacatggagctatgtttctgtgctgcaccagccgggaatcgaa

SEQ ID NO: 6 - DNA, 21 bp, synthetic construct
  source 1..21: mol_type = other DNA
tgttgtgtggaattgtgagcg

(19) United States
(12) Patent Application Publication
(10) Pub. No.: US 2025/0297274 A1
(43) Pub. Date: Sep. 25, 2025

(54) USE OF SOYBEAN GENE GLYMA.13G252100 IN REGULATING RESISTANCE OF SOYBEAN TO PHYTOPHTHORA

(71) Applicants: CENTER FOR AGRICULTURAL TECHNOLOGY, NORTHEAST INSTITUTE OF GEOGRAPHY AND AGROECOLOGY, Harbin (CN); NORTHEAST AGRICULTURAL UNIVERSITY, Harbin (CN)

(72) Inventors: Fanli MENG, Harbin (CN); Yang LI, Harbin (CN); Yong ZHANG, Harbin (CN); Xiaoming ZHANG, Harbin (CN); Wei ZHENG, Harbin (CN)

(21) Appl. No.: 19/231,598

(22) Filed: Jun. 9, 2025

Related U.S. Application Data
(63) Continuation of application No. PCT/CN2024/072392, filed on Jan. 15, 2024.

(51) Int. Cl. C12N 15/82; C12N 9/22; C12N 15/11

(52) U.S. Cl. CPC C12N 15/8279; C12N 9/226; C12N 15/111; C12N 15/8201; C12N 2310/20

(57) ABSTRACT

Provided is use of soybean gene Glyma.13G252100 in regulating resistance of soybean to Phytophthora. The nucleotide sequence of the soybean gene Glyma.13G252100 is set forth in SEQ ID NO: 1. The resistance of the soybean to Phytophthora is enhanced based on a decrease in an expression level of the soybean gene Glyma.13G252100; or the resistance of the soybean to Phytophthora is reduced based on an increase in an expression level of the soybean gene Glyma.13G252100. The present disclosure is the first discovery that modulation of Glyma.13G252100 expression can regulate resistance of soybean to Phytophthora.

### CROSS-REFERENCE TO RELATED APPLICATIONS

This application is a continuation of International Patent Application No. PCT/CN2024/072392 filed on Jan. 15, 2024, which is incorporated herein by reference in its entirety.

### STATEMENT REGARDING SEQUENCE LISTING

A Sequence Listing associated with this application is being filed concurrently herewith in XML format and is hereby incorporated by reference into the present specification. The file containing the Sequence listing is titled “Sequence_Listing.txt”, was created on Jun. 4, 2025, and is approximately 6,976 bytes in size.

### FIELD

The present disclosure belongs to the technical field of plant genetic engineering, and specifically relates to use of soybean gene Glyma.13G252100 in regulating resistance of soybean to Phytophthora. More specifically, the present disclosure relates to an sgRNA molecule, an expression vector, a reagent, a CRISPR/Cas9 system, a kit, and a method for regulating resistance of soybean to Phytophthora.

### BACKGROUND

Soybean (Glycine max) originated in China and is an important agricultural crop worldwide. It is widely cultivated as a major source of dietary protein and plant-based oil for human consumption. Additionally, it serves as a crucial protein source in animal feed and as a raw material in the chemical industry.

However, soybean is susceptible to various diseases, among which Phytophthora root rot is a significant one. Phytophthora root rot in soybean is an oomycete disease caused by Phytophthora sojae and is a destructive soil-borne disease characterized by fast progression, a broad host range, and significant challenges in effective control. Phytophthora sojae can infect soybean plants at any stage of growth. Its spores germinate most readily under conditions of high soil moisture and elevated temperatures. Initially, the motile zoospores of the pathogen are attracted by root exudates of soybean, such as daidzein and genistein, and subsequently invade plant cells via germ tubes. The pathogen then spreads throughout various parts of the soybean plant. This infection leads to symptoms such as leaf yellowing and water-soaked lesions on stems, which can severely reduce both yield and quality, and in extreme cases, may result in total crop loss. The main soybean cultivars currently in use are generally susceptible to this disease, making Phytophthora root rot a major limiting factor in improving soybean productivity.

Therefore, it is urgently needed to modify the expression of susceptibility genes in soybean to improve resistance of soybean to Phytophthora.

### SUMMARY

The present disclosure aims to solve, at least in part, one of the technical problems existing in the related art.

The inventors have discovered that in Arabidopsis thaliana, BIRI functions as a negative regulator of immune responses and cell death. Loss of BIRI leads to spontaneous cell death, sustained activation of defense responses, and severe dwarfism in plants. Similarly, silencing of the GmBIR1 gene (Glyma.18g246400), the soybean homolog of BIRI, also causes severe dwarfism in soybean plants and enhanced resistance to Soybean Mosaic Virus (SMV) and Pseudomonas syringae pv. glycinea, as well as cell death and sustained activation of immune responses. Consequently, enhanced resistance in the Glyma.18g246400-silenced soybean strain is accompanied by adversely affected normal plant growth. To overcome this issue, the inventors identified Glyma.13G252100, a homologous gene of Glyma.18g246400 in soybean, and employed gene editing techniques to knock out Glyma.13G252100. It was found that Glyma.13G252100 functions specifically as a negative regulator of immunity only during infection by Phytophthora sojae, and that mutation of this gene does not affect soybean yield.

To this end, in a first aspect of the present disclosure, the present disclosure provides use of soybean gene Glyma.13G252100 in regulating resistance of soybean to Phytophthora. The nucleotide sequence of the soybean gene Glyma.13G252100 is set forth in SEQ ID NO: 1. The present disclosure is the first discovery that modulation of Glyma.13G252100 expression can regulate resistance of soybean to Phytophthora.

In a second aspect of the present disclosure, the present disclosure provides a single guide RNA (sgRNA) molecule. According to an embodiment of the present disclosure, the sgRNA molecule includes at least one of nucleotide sequences set forth in SEQ ID NO: 2 or SEQ ID NO: 3. The sgRNA molecule according to the embodiment of the present disclosure exhibit low off-target rate and high editing efficiency, enabling partial or complete deletion of the soybean gene Glyma.13G252100 sequence, thereby enhancing resistance of soybean to Phytophthora.

In a third aspect of the present disclosure, the present disclosure provides an expression vector. According to an embodiment of the present disclosure, the expression vector carries the sgRNA molecule in the second aspect of the present disclosure and optionally a nucleic acid encoding a Cas9 molecule. The expression vector according to the embodiment of the present disclosure enables partial or complete deletion of the soybean gene Glyma.13 (252100 sequence, leading to partial or complete loss of the activity of the soybean gene Glyma.13G252100 and thus enhanced resistance of soybean to Phytophthora.

In a fourth aspect of the present disclosure, the present disclosure provides a reagent. According to an embodiment of the present disclosure, the reagent includes the sgRNA molecule in the second aspect of the present disclosure or the expression vector in the third aspect of the present disclosure. The reagent according to the embodiment of the present disclosure enables partial or complete deletion of the Glyma.13G252100 sequence, leading to partial or complete loss of the activity of the gene Glyma.13G252100 and thus enhanced resistance of soybean to Phytophthora.

In a fifth aspect of the present disclosure, the present disclosure provides a CRISPR/Cas9 system. According to an embodiment of the present disclosure, the CRISPR/Cas9 system includes the sgRNA molecule in the second aspect of the present disclosure and optionally a nucleic acid encoding a Cas9 molecule. The CRISPR/Cas9 system according to the embodiment of the present disclosure enables partial or complete deletion of the Glyma.13G252100 sequence, leading to partial or complete loss of the activity of the Glyma.13G252100 gene and thus enhanced resistance of soybean to Phytophthora.

In a sixth aspect of the present disclosure, the present disclosure provides a kit. According to an embodiment of the present disclosure, the kit includes the sgRNA molecule in the second aspect of the present disclosure or the expression vector in the third aspect of the present disclosure and optionally a nucleic acid encoding a Cas9 molecule. The kit according to the embodiment of the present disclosure enables partial or complete deletion of the Glyma.13G252100 sequence, leading to partial or complete loss of the activity of the Glyma.13G252100 gene and thus enhanced resistance of soybean to Phytophthora.

In a seventh aspect of the present disclosure, the present disclosure provides a method for regulating resistance of soybean to Phytophthora. According to an embodiment of the present disclosure, the method includes modifying the soybean gene Glyma.13G252100. The nucleotide sequence of the soybean gene Glyma.13G252100 is set forth in SEQ ID NO: 1. The method according to the embodiment of the present disclosure enables partial or complete deletion of the nucleotide sequence of the Glyma.13G252100 gene, leading to partial or complete loss of the activity of the Glyma.13G252100 gene and thus enhanced resistance of soybean to Phytophthora.

### BRIEF DESCRIPTION OF THE DRAWINGS

The above and/or additional aspects and advantages of the present disclosure will become more apparent and readily understood from the following description of the embodiments taken in conjunction with the accompanying drawings.

FIG. 1 shows qRT-PCR results of Glyma.13G252100 gene expression in major tissues in Example 1.

FIG. 2 shows the CRISPR/Cas9 knockout target sites and their corresponding positions in the Glyma.13G252100 gene in Example 1.

FIG. 3 shows the editing results of the A-H1 mutant at the target site in Example 2.

FIG. 4 is a diagram showing the differences in key agronomic traits between the wild-type recipient P3 and the knockout A-H1-T3 in Example 2.

FIG. 5 shows the results of Phytophthora resistance identification in Example 3 (Note: Panel A shows wild-type recipient P3 before infection; Panel B shows wild-type recipient P3 after infection; Panel C shows A-H1 before infection; Panel D shows A-H1 after infection).

### DETAILED DESCRIPTION

Embodiments of the present disclosure will be described in detail below. The embodiments described below are exemplary and are intended only to explain the present disclosure, and they should not be construed as limiting the present disclosure. Technical methods or conditions not specifically described in the embodiments may be implemented in accordance with techniques or conditions disclosed in the literature in the relevant field, or according to standard product manuals. Reagents or instruments not specified by manufacturer are all commercially available conventional products.

As used in the present disclosure, the terms “comprising,” “containing,” or “including” are open-ended expressions and are intended to cover the components explicitly mentioned as well as additional elements not expressly recited.

As used in the present disclosure, the terms “optionally,” “optional,” or “option” mean that the subsequently described event or circumstance may or may not occur, and that the description includes both instances where the event or circumstance occurs and where it does not.

As used in the present disclosure, the phrase “deletion, substitution, insertion, inversion, or translocation of at least a portion of the nucleotide sequence of the soybean gene Glyma.13G252100” refers to an alteration in the nucleotide sequence of the soybean gene Glyma.13G252100, thereby affecting the transcription and translation processes of the soybean gene Glyma.13G252100. Through the alteration of the nucleotide sequence of the soybean gene Glyma.13G252100, the expression of the soybean gene Glyma.13G252100 can be regulated. Forms of the alteration of the nucleotide sequence of the soybean gene Glyma.13G252100 gene include, but are not limited to, deletion, substitution, insertion, inversion, or translocation of the sequence.

As used in the present disclosure, the phrase “chemical modification of at least a portion of the nucleotide sequence of the soybean gene Glyma.13G252100” refers to the alteration of the properties, structure, and function of the nucleotide sequence of the soybean gene Glyma.13G252100, thereby affecting the transcription and translation processes of the soybean gene Glyma.13G252100, which in turn regulates the expression of the soybean gene Glyma.13G252100. Forms of the alteration of the properties, structure, and function of the nucleotide sequence of the soybean gene Glyma.13G252100 include, but are not limited to, methylation, phosphorylation, acetylation, or oxidation.

As used in the present disclosure, the term “methylation” refers to the addition of a methyl group (CH₃) to the Glyma.13G252100 gene, typically at cytosine (C) bases to form 5-methylcytosine. Methylation is an important epigenetic modification that plays a crucial role in gene expression regulation and cell differentiation.

As used in the present disclosure, the term “phosphorylation” refers to the addition of a phosphate group (PO₄⁻) to the Glyma.13G252100 gene, typically occurring on its deoxynucleotide components. Phosphorylation modification can affect the structure and function of DNA and is involved in processes such as DNA repair, recombination, and signal transduction.

As used in the present disclosure, the term “acetylation” refers to the addition of an acetyl group (CH₃CO) to the Glyma.13G252100 gene, typically occurring on histone proteins associated with the Glyma.13G252100 gene. Acetylation modification can affect chromatin structure and regulate gene expression.

As used in the present disclosure, “stably inherited” refers to the ability of a genetically edited mutant to transmit the mutation to its progeny in a stable genetic manner.

As used in the present disclosure, “gene editing”, also referred to as “genome editing” or “genome engineering”, is an emerging and precise genetic engineering technology that enables targeted modification of specific genes within the genome of an organism.

The present disclosure provides use of soybean gene Glyma.13G252100 in regulating resistance of soybean to Phytophthora, an sgRNA molecule, an expression vector, a reagent, a CRISPR/Cas9 system, a kit, and a method for regulating resistance of soybean to Phytophthora.

### Use of Soybean Gene Glyma.13G252100 in Regulating Resistance of Soybean to Phytophthora

In an aspect of the present disclosure, the present disclosure provides use of soybean gene Glyma.13G252100 in regulating resistance of soybean to Phytophthora. The nucleotide sequence of the soybean gene Glyma.13G252100 is set forth in SEQ ID NO: 1. The present disclosure is the first discovery that modulation of Glyma.13G252100 expression can regulate resistance of soybean to Phytophthora.

According to an embodiment of the present disclosure, the resistance of the soybean to Phytophthora is enhanced based on a decrease in an expression level of the soybean gene Glyma.13G252100.

According to an embodiment of the present disclosure, the resistance of the soybean to Phytophthora is reduced based on an increase in an expression level of the soybean gene Glyma.13G252100.

According to an embodiment of the present disclosure, the decrease in the expression level of the soybean gene Glyma.13G252100 is achieved by at least one of:

a gene editing system or RNA interference.

According to an embodiment of the present disclosure, the gene editing system includes at least one of a CRISPR-Cas9 system, CRISPR-Cas12a/Cpf1, base editors, TALEN, or ZFN.

According to an embodiment of the present disclosure, the CRISPR-Cas9 system includes an sgRNA.

The sgRNA has at least one of nucleotide sequences set forth in SEQ ID NO: 2 or SEQ ID NO: 3.

According to an embodiment of the present disclosure, the increase in the expression level of the soybean gene Glyma.13G252100 is achieved by overexpressing the soybean gene Glyma.13G252100.

### SgRNA Molecule

In a second aspect of the present disclosure, the present disclosure provides an sgRNA molecule. According to an embodiment of the present disclosure, the sgRNA molecule includes at least one of nucleotide sequences set forth in SEQ ID NO: 2 or SEQ ID NO: 3. The sgRNA molecule according to the embodiment of the present disclosure exhibit low off-target rate and high editing efficiency, enabling partial or complete deletion of the soybean gene Glyma.13G252100 sequence, thereby enhancing resistance of soybean to Phytophthora.

According to an embodiment of the present disclosure, the sgRNA molecule includes the nucleotide sequence set forth in SEQ ID NO: 2.

According to an embodiment of the present disclosure, the sgRNA molecule includes the nucleotide sequence set forth in SEQ ID NO: 3.

According to an embodiment of the present disclosure, the sgRNA molecule includes the nucleotide sequence set forth in SEQ ID NO: 2 and the nucleotide sequence set forth in SEQ ID NO: 3.

### Expression Vector

In a third aspect of the present disclosure, the present disclosure provides an expression vector. According to an embodiment of the present disclosure, the expression vector carries the sgRNA molecule in the second aspect of the present disclosure and optionally a nucleic acid encoding a Cas9 molecule. The expression vector according to the embodiment of the present disclosure enables partial or complete deletion of the soybean gene Glyma.13G252100 sequence, leading to partial or complete loss of the activity of the soybean gene Glyma.13G252100 and thus enhanced resistance of soybean to Phytophthora.

### Reagent

In a fourth aspect of the present disclosure, the present disclosure provides a reagent. According to an embodiment of the present disclosure, the reagent includes the sgRNA molecule in the second aspect of the present disclosure or the expression vector in the third aspect of the present disclosure. The reagent according to the embodiment of the present disclosure enables partial or complete deletion of the Glyma.13G252100 sequence, leading to partial or complete loss of the activity of the gene Glyma.13G252100 and thus enhanced resistance of soybean to Phytophthora.

### CRISPR/Cas9 System

In a fifth aspect of the present disclosure, the present disclosure provides a CRISPR/Cas9 system. According to an embodiment of the present disclosure, the CRISPR/Cas9 system includes the sgRNA molecule in the second aspect of the present disclosure and optionally a nucleic acid encoding a Cas9 molecule. The CRISPR/Cas9 system according to the embodiment of the present disclosure enables partial or complete deletion of the Glyma.13G252100 sequence, leading to partial or complete loss of the activity of the Glyma.13G252100 gene and thus enhanced resistance of soybean to Phytophthora.

### Kit

In a sixth aspect of the present disclosure, the present disclosure provides a kit. According to an embodiment of the present disclosure, the kit includes the sgRNA molecule in the second aspect of the present disclosure or the expression vector in the third aspect of the present disclosure and optionally a nucleic acid encoding a Cas9 molecule. The kit according to the embodiment of the present disclosure enables partial or complete deletion of the Glyma.13G252100 sequence, leading to partial or complete loss of the activity of the Glyma.13G252100 gene and thus enhanced resistance of soybean to Phytophthora.

### Method for Regulating Resistance of Soybean to Phytophthora

In a seventh aspect of the present disclosure, the present disclosure provides a method for regulating resistance of soybean to Phytophthora. According to an embodiment of the present disclosure, the method includes modifying the soybean gene Glyma.13G252100. The nucleotide sequence of the soybean gene Glyma.13G252100 is set forth in SEQ ID NO: 1. The method according to the embodiment of the present disclosure enables partial or complete deletion of the nucleotide sequence of the Glyma.13G252100 gene, leading to partial or complete loss of the activity of the Glyma.13G252100 gene and thus enhanced resistance of soybean to Phytophthora.

According to an embodiment of the present disclosure, said modifying is achieved by:
at least one of deletion, substitution, insertion, inversion, or translocation of at least a portion of the nucleotide sequence of the soybean gene Glyma.13G252100; or
chemical modification of at least a portion of the nucleotide sequence of the soybean gene Glyma.13G252100.

According to an embodiment of the present disclosure, the chemical modification includes at least one of methylation, phosphorylation, or acetylation.

According to an embodiment of the present disclosure, said modifying refers to a decrease in an expression level of the soybean gene Glyma.13G252100, and said regulating refers to enhancing the resistance of the soybean to Phytophthora.

According to an embodiment of the present disclosure, said modifying refers to an increase in an expression level of the soybean gene Glyma.13G252100, and said regulating refers to reducing the resistance of the soybean to Phytophthora. According to the method in an embodiment of the present disclosure, a model of Phytophthora root rot in soybean is constructed and is used for screening therapeutic agents for treating Phytophthora root rot in soybean.

According to an embodiment of the present disclosure, the decrease in the expression level of the soybean gene Glyma.13G252100 is achieved by at least one of a gene editing system or RNA interference.

According to an embodiment of the present disclosure, the gene editing system includes at least one of a CRISPR-Cas9 system, CRISPR-Cas12a/Cpf1, base editors, TALEN, or ZFN.

According to an embodiment of the present disclosure, the gene editing system includes the sgRNA molecule in a second aspect of the present disclosure, the expression vector in a third aspect of the present disclosure, or the reagent in a fourth aspect of the present disclosure.

According to an embodiment of the present disclosure, the gene editing system is selected from the CRISPR/Cas9 system in a fifth aspect of the present disclosure.

According to an embodiment of the present disclosure, the increase in the expression level of the soybean gene Glyma.13G252100 is achieved by overexpressing the soybean gene Glyma.13G252100.

According to an embodiment of the present disclosure, said overexpressing the soybean gene Glyma.13G252100 is achieved by introducing an expression vector including an overexpressed soybean gene Glyma.13G252100 into soybean genome.

The sequence list of the present disclosure is as follows:

| SEQ ID | Sequence | |
|---|---|---|
| NO: | Name | Sequence |
| SEQ ID | Glyma.13G2 | ATGGTCTACTATGCCCCTTGGACAGAAT |
| NO: 1 | 52100 | TCAAAAAGAGGAGGAGTAACGCTATTC |
| | | ATCCCTTAATGAAGAAGCAGAACCATC |
| | | AAAAGAACACTAGACAAGAAGCTGAAT |
| | | TGCTACCACTAGCCATGCAAGAGCAAG |
| | | GAAGCAAAGAGGTACTCTCTCTCTCTC |
| | | TCTCTCTCTCTCTCTCTGTTTCTCTAT |
| | | TGATCAGTTGTGAGCACATGATATTTG |
| | | AGATCAAACACTGATGTCATTACTTTA |
| | | AGGTCACGTCACGTATTTATTTTCAAA |
| | | TTTTTATGTCCATGATATATCAAGATT |
| | | GTGACATATATTTCATCTAAGCTATCA |
| | | AGATTGTTGGAGAGATTGATTCCTAGA |
| | | ATGAGCTTCAAGGAATTATGCAAGGCA |
| | | ACCGATTACTTTAGCACAGATAACGTT |
| | | TTAGGGATAGGAACGACAGGGATTATG |
| | | TACAAGGCAAAGGTACCAAATAACTGT |
| | | TTTCTAGCAGTTAAGAGACTATATGGC |
| | | GCTGATGAATACAAGAGGGAATTCTTG |
| | | CTTGAAACAATGATTCTGGGGAGACAC |
| | | AGGCACAGAAACATAGCTCCATTGGTA |
| | | GGATTCTGCATAGAAAAAAGAGAAAGG |
| | | ATTTTGGTGTATAAGTACATGTCTAAT |
| | | GGAAGACTCAGTGATTGGTTCCATTCT |
| | | GATGAAGGAGATCAAAAAATAAAACTA |
| | | GAATGGCCAGAGAGAATTCACATTGCA |
| | | CTTGGGATTGCAAGAGGTTTATCTTGG |
| | | CTCCACAAAAGGTGCAAGATATTCCAT |
| | | CTTAATTTAGATTCAGAGTGTGTCTTG |
| | | CTAGACAGGTATTTTGAGCCAAAAATA |
| | | TCCAATTTTGAGAAGGCAAAATTTCTA |
| | | AACCATACAGTTGAAGATCATGTGAGG |
| | | ATGAATGTTATGAATTAG |
| SEQ ID | Glyma.13G2 | GAGCTTCAAGGAATTATGCA |
| NO: 2 | 52100-sg1 | |
| SEQ ID | Glyma.13G2 | GCACAGAAACATAGCTCCAT |
| NO: 3 | 52100-sg2 | |
| SEQ ID | Glyma.13G2 | TGGTCTCGTGCAGAGCTTCAAGGAATT |
| NO: 4 | 52100-sg1-F | ATGCAGTTTTAGAGCTAGAAATAGC |
| SEQ ID | Glyma.13G2 | TGGTCTCGAAACATGGAGCTATGTTTC |
| NO: 5 | 52100-sg1-R | TGTGCTGCACCAGCCGGGAATCGAA |
| SEQ ID | STU-TEST- | TGTTGTGTGGAATTGTGAGCG |
| NO: 6 | 4R | |

The following examples are provided to further illustrate the solutions of the present disclosure. Those skilled in the art will understand that the following examples are intended only to explain the present disclosure, and they should not be construed as limiting the scope of the present disclosure. Technical methods or conditions not specifically described in the embodiments may be implemented in accordance with techniques or conditions disclosed in the literature in the relevant field, or according to standard product manuals. Reagents or instruments not specified by manufacturer are all commercially available conventional products.

### Example 1

(1) Screening of Homologous Genes of GmBIR1 (Glyma.18g246400) in Soybeans

In this example, the sequence of GmBIR1 gene (Glyma.18g246400) was used to conduct a search on the Phytozome v13 database, and gene sequences with more than 60% homology to GmBIR1 (Glyma.18g246400) were selected as candidate sequences. These candidate homologous sequences were aligned with the Arabidopsis thaliana AtBIR1 genome sequence. As a result, Glyma.13G252100 was identified as a homologous gene of GmBIR1 (Glyma.18g246400) in soybean, as shown in Table 1.

TABLE 1
Homology Alignment of GmBIR1 (Glyma.18g246400)

| | Homology | |
|---|---|---|
| Gene name | GmBIR1-1 Glyma.18G246400 | AtBIR1 AT5G48380 |
| Glyma.18G246400 | 100.00% | 78.10% |
| Glyma.13G252100 | 66.00% | 60.70% |

### (2) Expression Pattern Analysis of Glyma.13G252100 Gene

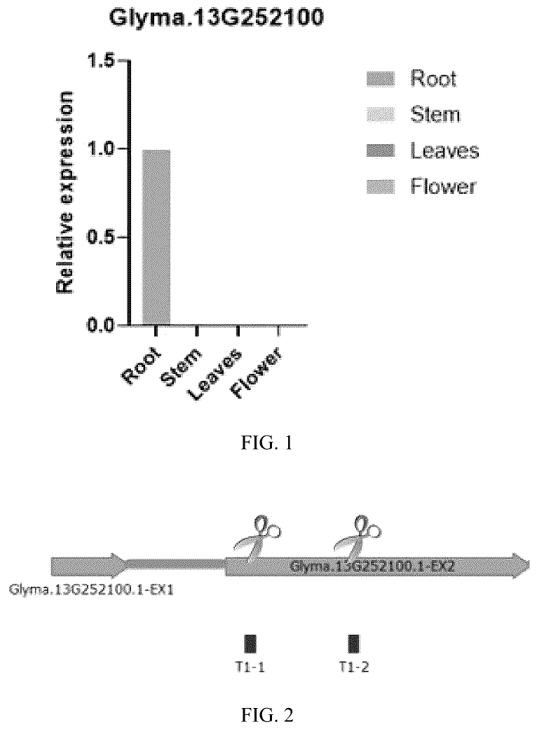

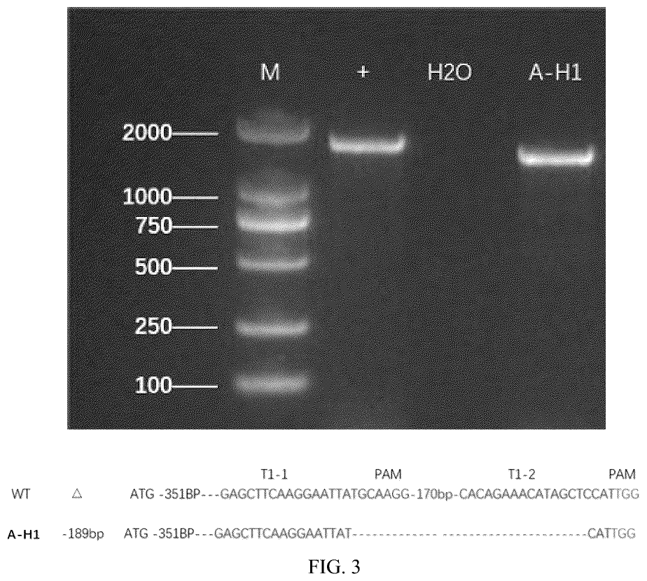

Based on soybean transcriptome data from the Phytozome v13 database, the expression levels of the Glyma.13G252100 gene was analyzed across various tissues including root, stem, leaf, and flower. The data indicated that the Glyma.13G252100 gene was specifically expressed in soybean roots, but at a very low level. RT-PCR was further used to analyze the expression level of the Glyma.13G252100 gene. The results, as shown in FIG. 1, were consistent with the Phytozome database data, further confirming that under normal growth conditions, Glyma.13G252100 was specifically expressed in soybean roots, but at a very low level.

### Example 2

In this example, the website benchling.com was used to design gRNA target sites T1-1 (primer for T1-1 target site: Glyma.13G252100-sg1, sequence set forth in SEQ ID NO: 2) and T1-2 (primer for T1-2 target site: Glyma.13G252100-sg2, sequence set forth in SEQ ID NO: 3) on the exons of the Glyma.13G252100 gene. Based on these, single-target knockout vectors Glyma.13G252100-KO-T1-1 and Glyma.13G252100-KO-T1-2, as well as a dual-target knockout vector Glyma.13G252100-KO-T1-1-T1-2 were constructed for Glyma.13G252100-KO, respectively.

### 2.1 Construction Process of Single-Target Knockout Vectors Glyma.13G252100-KO-T1-1 and Glyma.13G252100-KO-T1-2

Using the pGES401-single-target empty vectors (Glyma.13G252100-sg1 and Glyma.13G252100-sg2) as the template, a tRNA-sgRNA scaffold-tRNA structure was amplified by PCR. The PCR products were recovered by gel extraction and then ligated with the pGES401 vector. The ligation products were transformed into competent E. coli DH5a cells. Positive clones were identified by colony PCR and sent for sequencing. Plasmids with correct sequencing results were extracted and transformed into Agrobacterium K599. Colonies confirmed by PCR and sequencing indicated successful construction of the single-target CRISPR/Cas9 knockout vectors Glyma.13G252100-KO-T1-1 and Glyma.13G252100-KO-T1-2 for Glyma.13G252100. These vectors carried the Bar selectable marker gene (conferring glufosinate resistance).

### 2.2 Construction Process of Dual-Target Knockout Vector Glyma.13G252100-KO-T1-1-T1-2

Using the pGES401-dual-target empty vector (Glyma.13G252100-sg1 and Glyma.13G252100-sg2) as the template, an additional tRNA-sgRNA-sgRNA scaffold-tRNA structure was amplified by PCR. A DNA fragment with two sgRNAs in tandem (i.e., dual-target Glyma.13G252100-KO-T1-1-T1-2) was constructed using the Golden Gate method. The specific steps were as follows:
(1) PGES401-Glyma.13G252100-sg1 and PGES401-Glyma.13G252100-sg2 were obtained through ligation reactions. The ligation system included: 5 μL of PGES401 plasmid, 1 μL of T4 DNA Ligase, 2 μL of T4 DNA Ligase Buffer, 2 μL of BsaI, 1 μL of Glyma.13G252100-sg1, 1 μL of Glyma.13G252100-sg2, and 5 μL of ddH₂O.
(2) A DNA fragment of tRNA-sgRNA-sgRNA Scaffold-tRNA was amplified by PCR. The PCR reaction conditions included: (37° C. for 5 min, 16° C. for 5 min)×25 cycles, 37° C. for 15 min, and 85° C. for 5 min. The amplification primers included: Glyma.13G252100-sg1-F (protective bases+BsaI recognition site+Glyma.13G252100-sg1+sgRNA scaffold, nucleotide sequence set forth in SEQ ID NO: 4), Glyma.13G252100-sg1-R (protective bases+BsaI recognition site+BsaI cleavage site+Glyma.13G252100-sg2+sgRNA scaffold, nucleotide sequence set forth in SEQ ID NO: 5).
(3) A DNA fragment of tRNA-sgRNA-sgRNA Scaffold-tRNA obtained above was amplified by PCR using the high-fidelity enzyme KOD One™ PCR master Mix (purchased from TOYOBO) to obtain PCR products containing the DNA fragment with two sgRNAs in tandem. The PCR reaction conditions included: pre-denaturation at 98° C. for 3 min, (denaturation at 98° C. for 15 s, annealing at 58° C. for 15 s, extension at 68° C. for 20 s)×35 cycles, and then extension at 68° C. for 5 min. The resulting PCR products were recovered by gel extraction.
(4) The recovered DNA fragment and pGES401 vector were added to the ligation reaction system and the obtained ligation product was transformed into competent E. coli DH5a cells. Positive clones were identified by colony PCR and sent for sequencing using primer STU-TEST-4R (nucleotide sequence set forth in SEQ ID NO: 6). Sequencing results confirmed that the pGES401 vector contained the sequence of tRNA, Glyma.13G252100-sg1, sgRNA scaffold, tRNA, Glyma.13G252100-sg2, sgRNA scaffold, and tRNA. The correctly sequenced plasmid was extracted and transformed into Agrobacterium K599. Colonies confirmed by PCR and sequencing indicated successful construction of the dual-target knockout vector Glyma.13G252100-KO-T1-1-T1-2, which also carried the Bar selectable marker gene (glufosinate resistance).

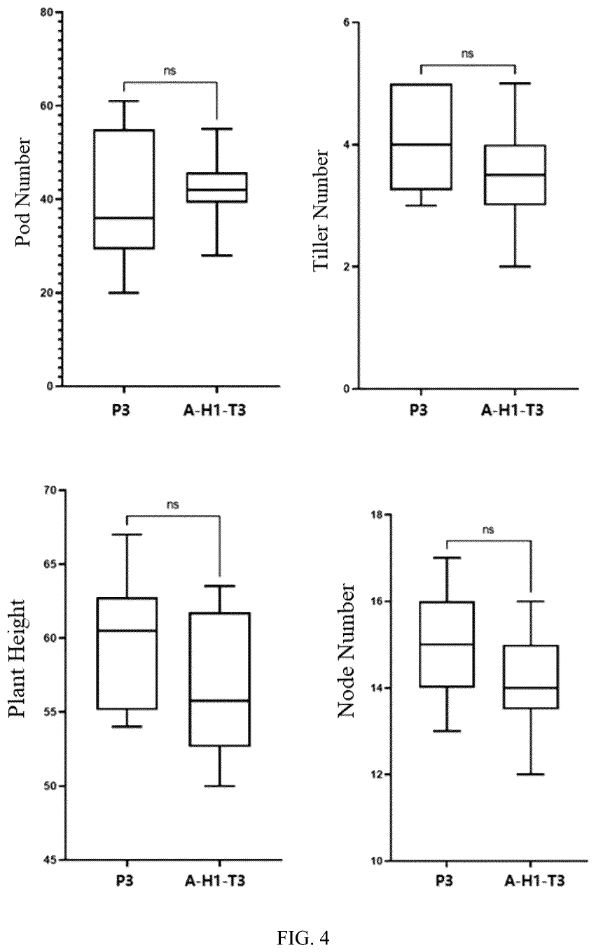

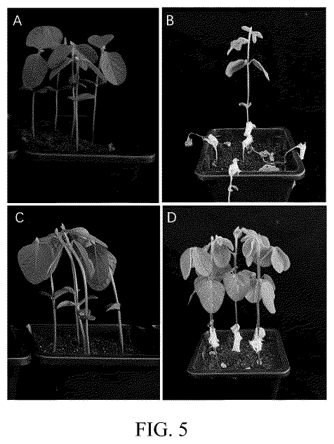

The constructed vectors Glyma.13G252100-KO-T1-1, Glyma.13G252100-KO-T1-2, and Glyma.13G252100-KO-T1-1-T1-2 were each transformed into Agrobacterium EHA101 and used to transform the recipient soybean P3 via the cotyledonary node method. Mutants with large fragment deletions were obtained: A-H1 (a mutant corresponding dual-target constructed to the knockout vector Glyma.13G252100-KO-T1-1-T1-2), A-H2 (a mutant constructed corresponding to the single-target knockout vector Glyma.13G252100-KO-T1-1), and A-H3 (a mutant constructed corresponding to the single-target knockout vector Glyma.13G252100-KO-T1-2). Mutant A-H1 (a mutant constructed corresponding to the dual-target knockout vector Glyma.13G252100-KO-T1-1-T1-2) was exemplified in this example. As illustrated in FIG. 3, both T1-1 and T1-2 target sites were cleaved in mutant A-H1, resulting in a 189 bp deletion of the Glyma.13G252100 gene. Subsequently, agronomic traits such as plant height, node number, and tiller number were investigated in the wild-type recipient P3 and the T3-generation homozygous mutant A-H1 (also referred to as A-H1-T3). The results, as illustrated in FIG. 4, showed that compared with the wild-type recipient P3, the knockout strain A-H1-T3 exhibited slightly reduced plant height and node number without statistically significant differences, as well as tiller number and pod number per plant that showed no significant differences from wild-type recipient P3.

### Example 3

In this example, two Phytophthora-resistant soybean cultivars from Heilongjiang Province, China, Heihe 32 and Hefeng 50, were selected as resistant controls, while Heihe 38, a susceptible cultivar, was used as a susceptible control. In addition, the wild-type recipient P3 and the knockout mutant A-H1-T3 were used as test samples. Five seeds were sown evenly per pot, with four replicates. After 7 days of cultivation under identical conditions, inoculation was carried out using the hypocotyl-wounding method. Inoculated seedlings were then incubated in a constant temperature and humidity chamber at 35° C. and 80% relative humidity. Disease symptoms were assessed 7 days post-inoculation. The results showed that the susceptible control Heihe 38 exhibited a mortality rate of over 70%, confirming the validity of this disease resistance evaluation. According to the evaluation results shown in Table 2 and FIG. 5, the wild-type recipient P3 had a mortality rate comparable to that of Heihe 38, and was classified as susceptible. In contrast, the A-H1-T3 mutant was identified as resistant, exhibiting significantly enhanced resistance to Phytophthora compared to wild-type P3. These results demonstrate that the Glyma.13G252100 gene acts as a negative regulator of soybean resistance to Phytophthora.

TABLE 2
Evaluation of Soybean Resistance to Phytophthora Root Rot

| Variety | Number of | Number of | Plant | Resistance |
|---|---|---|---|---|
| Number | Inoculated Plants | Dead Plants | Mortality | Evaluation |
| Heihe 32 | 20 | 6 | 30% | Resistant (R) |
| Hefeng 50 | 20 | 4 | 20% | Resistant (R) |
| Heihe 38 | 20 | 16 | 80% | Susceptible (S) |
| Recipient P3 | 20 | 17 | 85% | Susceptible (S) |
| A-H1-T3 | 20 | 4 | 20% | Resistant (R) |

In the description of the present specification, reference to the terms such as “an embodiment,” “some embodiments,” “an example,” “a specific example,” or “some examples” means that a particular feature, structure, material, or characteristic described in connection with the embodiment or example is included in at least one embodiment or example of the present disclosure. The illustrative expressions of the above terms as used in this specification do not necessarily refer to the same embodiment or example. Furthermore, the particular features, structures, materials, or characteristics described may be combined in any suitable manner in one or more embodiments or examples. In addition, unless otherwise conflicting, those skilled in the art will appreciate that the different embodiments or examples described in this specification, as well as the features of different embodiments or examples, may be combined or integrated.

Although the embodiments of the present disclosure have been shown and described above, it will be understood by those of ordinary skill in the art that the above embodiments are exemplary only and should not be construed as limiting the scope of the invention. Modifications, alterations, substitutions, and variations can be made to the described embodiments without departing from the scope of the present disclosure.

What is claimed is:

1. An sgRNA molecule, comprising at least one of nucleotide sequences set forth in SEQ ID NO: 2 or SEQ ID NO: 3.

2. An expression vector, carrying:
the sgRNA molecule according to claim 1; and
optionally, a nucleic acid encoding a Cas9 molecule.

3. A reagent, comprising the sgRNA molecule according to claim 1.

4. A CRISPR/Cas9 system, comprising:
the sgRNA molecule according to claim 1, and
optionally, a nucleic acid encoding a Cas9 molecule.

5. A kit, comprising:
the sgRNA molecule according to claim 1; and
optionally, a nucleic acid encoding a Cas9 molecule.

6. A method for regulating resistance of soybean to Phytophthora, the method comprising:
modifying soybean gene Glyma.13G252100,
wherein the nucleotide sequence of the soybean gene Glyma.13G252100 is set forth in SEQ ID NO: 1.

7. The method according to claim 6, wherein said modifying is achieved by:
at least one of deletion, substitution, insertion, inversion, or translocation of at least a portion of the nucleotide sequence of the soybean gene Glyma.13G252100; or
chemical modification of at least a portion of the nucleotide sequence of the soybean gene Glyma.13G252100, optionally, the chemical modification comprising at least one of methylation, phosphorylation, or acetylation.

8. The method according to claim 6, wherein:
said modifying refers to a decrease in an expression level of the soybean gene Glyma.13G252100, and said regulating refers to enhancing the resistance of the soybean to Phytophthora; or
said modifying refers to an increase in an expression level of the soybean gene Glyma.13G252100, and said regulating refers to reducing the resistance of the soybean to Phytophthora.

9. The method according to claim 8, wherein the decrease in the expression level of the soybean gene Glyma.13G252100 is achieved by at least one of a gene editing system or RNA interference.

10. The method according to claim 9, wherein the gene editing system comprises at least one of a CRISPR-Cas9 system, CRISPR-Cas12a/Cpf1, base editors, TALEN, or ZFN.

11. The method according to claim 10, wherein the gene editing system comprises an sgRNA molecule, the sgRNA molecule comprising at least one of nucleotide sequences set forth in SEQ ID NO: 2 or SEQ ID NO: 3.

12. The method according to claim 11, wherein the gene editing system is an CRISPR/Cas9 system comprising the sgRNA molecule and a nucleic acid encoding a Cas9 molecule.

13. The method according to claim 8, wherein the increase in the expression level of the soybean gene Glyma.13G252100 is achieved by overexpressing the soybean gene Glyma.13G252100.

14. The method according to claim 13, wherein said overexpressing the soybean gene Glyma.13G252100 is achieved by introducing an expression vector comprising an overexpressed soybean gene Glyma.13G252100 into soybean genome.
